(19) United States
(12) Patent Application Publication
(10) Pub. No.: US 2011/0230466 A1
(43) Pub. Date: Sep. 22, 2011

(54) Association of a sinus node if current inhibitor and calcium inhibitor and pharmaceutical compositions containing it

(75) Inventors: Vidal Benatar, Saint-Cloud (FR); Guy Lerebours-Pigeonniere, Levallois Perret (FR)

(73) Assignee: LES LABORATOIRES SERVIER, Suresnes Cedex (FR)

(21) Appl. No.: 13/068,551

(22) Filed: May 13, 2011

Related U.S. Application Data
(63) Continuation of application No. 12/386,687, filed on Apr. 22, 2009, now abandoned.
Application No. 12/386,687 is a continuation of application No. 11/643,046, filed on Dec. 20, 2006, now abandoned.

(30) Foreign Application Priority Data
Dec. 21, 2005 (FR) 05.13008

(51) Int. Cl. A61K 31/55; A61P 9/00

(52) U.S. Cl. 514/212.07

(57) ABSTRACT

Association comprising a selective and specific sinus node I_{f} current inhibitor, more especially ivabradine, and a calcium inhibitor.

Medicinal products containing the same which are useful in treating angina.

The present invention relates to a new association of a selective and specific sinus node I_{f} current inhibitor and a calcium inhibitor.

More specifically, the present invention relates to a new association of a selective and specific sinus node I_{f} current inhibitor which is ivabradine, or 3-{3-[{[(7S)-3,4-dimethoxy-bicyclo[4.2.0]octa-1,3,5-trien-7-yl]methyl}(methyl)amino]propyl}-7,8-dimethoxy-1,3,4,5-tetrahydro-2H-3-benzazepin-2-one, of formula (I):

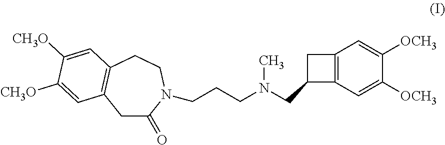

and its hydrates and crystalline forms and addition salts thereof with a pharmaceutically acceptable acid, and a calcium inhibitor, more especially a calcium inhibitor from the dihydropyridine class.

Selective and specific sinus node I_{f} current inhibitors, more especially ivabradine and its hydrates and crystalline forms and addition salts thereof with a pharmaceutically acceptable acid, more especially its hydrochloride, have very valuable pharmacological and therapeutic properties, especially negative chronotropic properties (lowering of heart rate), which make these compounds useful in the treatment or prevention of various clinical situations of myocardial ischaemia such as angina pectoris, myocardial infarct and associated rhythm disturbances, and also in various pathologies involving rhythm disturbances, especially supraventricular rhythm disturbances, and in heart failure.

The preparation and therapeutic use of ivabradine and addition salts thereof with a pharmaceutically acceptable acid, more especially its hydrochloride, have been described in the European patent specification EP 0 534 859.

The Applicant has now discovered, surprisingly, that selective and specific sinus node I_{f} current inhibitors, more especially ivabradine, or 3-{3-[{[(7S)-3,4-dimethoxybicyclo-[4.2.0]octa-1,3,5-trien-7-yl]methyl}(methyl)amino]propyl}-7,8-dimethoxy-1,3,4,5-tetra-hydro-2H-3-benzazepin-2-one, used in association with a calcium inhibitor, more especially a calcium inhibitor from the dihydropyridine class, have very valuable properties which allow them to be used in association in the treatment of angina.

Calcium inhibitors are compounds which have the fundamental property of blocking the permeability of certain channels of the cell membrane to calcium. They prevent opening of the pores of the voltage-dependent channels and accordingly oppose the entry of calcium into vascular smooth muscle fibres. They lower the level of free intracellular calcium, which has the consequence of reducing the smooth muscle tone of the peripheral and coronary vessels. Accordingly, these compounds and, more especially, those belonging to the dihydropyridine class are especially indicated in the treatment of angina because they reduce venous return, thereby reducing the work load on the left ventricle, and they lower myocardial oxygen consumption on the one hand and improve coronary blood flow by virtue of their vasodilatory action on the large epicardial arteries on the other hand. One of the consequences of the peripheral vasodilatory effect of the dihydropyridines is to bring about a reflex tachycardia which can persist in patients being treated for angina pectoris. It is known that there exists a strong relationship between an increase in heart rate and cardiovascular mortality in the coronary patient. This has to be linked with the possible risk of increased cardiovascular mortality and of myocardial infarcts reported in respect of the dihydropyridines.

The adverse effects most frequently encountered with the dihydropyridines are tachycardia, palpitations, headaches and oedemas of the lower limbs, which are dose-dependent.

There is accordingly a real need for new treatments that make it possible to benefit from the positive effects of those compounds whilst increasing their safety margin, especially their cardiovascular safety margin.

The Applicant has now discovered, surprisingly, that selective and specific sinus node I_{f} current inhibitors, more especially ivabradine, are not only capable of potentiating the effects of calcium inhibitors and, more especially, those belonging to the dihydropyridine class, but have moreover shown themselves to be excellently capable of improving the safety profile of those calcium antagonists and, more especially, the adverse cardiac effects, oedemas of the lower limbs and headaches. This double effect makes it possible to consider use of the association according to the invention in the treatment of angina with increased safety of use.

The selective and specific sinus node I_{f} current inhibitors in the association according to the invention are, more particularly, ivabradine, zatebradine and cilobradine, and also their hydrates, crystalline forms and addition salts with a pharmaceutically acceptable acid or base.

The calcium inhibitors in the association according to the invention are, more particularly, those belonging to the dihydropyridine class. Without implying any limitation, the calcium inhibitors in the association according to the invention are: amlodipine, nifedipine, felodipine and their hydrates, crystalline forms and addition salts with a pharmaceutically acceptable acid or base and, more especially, the besylate or maleate.

The invention relates more especially to the association between ivabradine or one of its hydrates, crystalline forms and addition salts with a pharmaceutically acceptable acid, more especially its hydrochloride, and a calcium inhibitor or one of its hydrates, crystalline forms and addition salts with a pharmaceutically acceptable acid.

Even more preferably, the invention relates to the association between ivabradine or one of its hydrates, crystalline forms and addition salts with a pharmaceutically acceptable acid, more especially its hydrochloride, and amlodipine or one of its hydrates, crystalline forms and addition salts with a pharmaceutically acceptable acid, more especially its besylate or maleate.

The invention relates also to pharmaceutical compositions comprising the association of a selective and specific sinus node I_{f} current inhibitor and a calcium inhibitor, in combination with one or more pharmaceutically acceptable excipients.

The invention relates more especially to pharmaceutical compositions comprising the association of a selective and specific sinus node I_{f} current inhibitor which is ivabradine or its hydrates, crystalline forms or addition salts with a pharmaceutically acceptable acid, more especially its hydrochloride, and a calcium inhibitor, more especially a calcium inhibitor from the dihydropyridine class, in combination with one or more pharmaceutically acceptable excipients.

Amongst the pharmaceutical compositions according to the invention, there may be mentioned, more especially, those that are suitable for oral, parenteral or nasal administration, tablets, dragées, sublingual tablets, capsules, lozenges, suppositories, creams, ointments, dermal gels etc. and also pharmaceutical compositions having programmed, delayed, prolonged or deferred release.

Besides the selective and specific sinus node I_{f} current inhibitor and the calcium inhibitor, the pharmaceutical compositions according to the invention comprise one or more excipients or carriers selected from diluents, lubricants, binders, disintegration agents, absorbents, colourants, sweeteners etc.

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the sex, age and weight of the patient, the administration route, the nature of the disorder and of any associated treatments and ranges from 1 to 500 mg of ivabradine per 24 hours and, more preferably, from 15 to 20 mg per day and, also preferably, from 5 to 15 mg per day. The dose of the calcium inhibitor may be less than that used when it is administered on its own.

The following Examples illustrate the invention but do not limit it in any way.

### Pharmaceutical Compositions:

Preparation formula for 1000 tablets each containing 10 mg of ivabradine and 5 mg of amlodipine:

| | |
|---|---|
| Ivabradine hydrochloride | 10 g |
| Amlodipine besylate | 5 g |
| Lactose monohydrate | 62 g |
| Magnesium stearate | 1.3 g |
| Povidone | 9 g |
| Anhydrous colloidal silica | 0.3 g |
| Cellulose sodium glycolate | 30 g |
| Stearic acid | 2.6 g |

Other examples of pharmaceutical compositions according to the invention are given hereinbelow, without implying any limitation:

### EXAMPLE 1

| Constituents | Amount (mg) |
|---|---|
| ivabradine | 10 |
| amlodipine | 5 |

### EXAMPLE 2

| Constituents | Amount (mg) |
|---|---|
| ivabradine | 15 |
| amlodipine | 5 |

### EXAMPLE 3

| Constituents | Amount (mg) |
|---|---|
| ivabradine | 10 |
| amlodipine | 10 |

### EXAMPLE 4

| Constituents | Amount (mg) |
|---|---|
| ivabradine | 15 |
| amlodipine | 10 |

The dosage for the pharmaceutical compositions described above consists of the administration, per os, of one tablet per 24 hours.

In the populations at risk, corresponding to patients who are hypertensive and more than 75 years old, the initial critical dose administered by the oral route is 5 mg of ivabradine and 5 mg of amlodipine per 24 hours in the form of a tablet.

### Clinical Stud:

Two clinical studies carried out in patients being treated with dihydropyridine type calcium antagonists who still complained of painful angina pectoris attacks (despite the calcium antagonist) have shown that concomitant treatment with ivabradine makes it possible to reduce these attacks very substantially (by about 60%).

TABLE 1
Change in the number of angina attacks in patients being
administered dihydropyridines on inclusion and having
been administered ivabradine for 1 year.
Number of angina pectoris attacks (per week)

| | On inclusion | After treatment | % |
|---|---|---|---|
| Study 019: | 1.9 ± 2.8 | 0.7 ± 1.3 | −61.4 |
| n = 27 | | | |
| Study 021: | 2.2 ± 3.4 | 0.9 ± 2.9 | −58.9 |
| n = 114 | | | |

n = number of patients

In addition, in surprising manner, the association of ivabradine with amlodipine brought about an improvement in the safety and acceptability profile of the amlodipine. In the course of the clinical development of ivabradine for the treatment of angina pectoris, studies on the acceptability of ivabradine were carried out compared to amlodipine monotherapy or amlodipine in association with ivabradine. The results show that when ivabradine is associated with amlodipine, the safety of use of the latter, especially its cardiac safety, increases:

TABLE 2
Adverse events in coronary patients treated
with amlodipine alone or with the association
amlodipine + ivabradine, per 100 patient-years of exposure

| | Ivabradine + | Amlodipine |
|---|---|---|
| | calcium | alone |
| | antagonists n = 686 | n = 401 |
| | Patient-years: | Patient-years: |
| | 262.6 | 94.8 |
| Adverse cardiac events | 40.0 | 58.0 |
| Cardiac arrhythmias | 28.6 | 35.9 |
| Unstable angina | 2.3 | 5.3 |
| Myocardial infarct | 1.9 | 3.2 |
| Deterioration of coronary | 0.4 | 2.1 |
| disease | | |
| Palpitations | 1.1 | 3.1 |
| Oedemas of the lower limbs | 22.9 | 33.5 |
| Headaches | 3.8 | 9.5 |

n = number of patients

A lower level of adverse events can clearly be seen when ivabradine is added to the amlodipine, especially for cardiac events of the cardiac arrhythmia type and the coronary ischaemic event type (unstable angina, myocardial infarct and deterioration of coronary disease). It is important to note that the incidence of oedemas of the lower limbs, which are the most frequent adverse effect for amlodipine and the cause of cessation of treatment in close to 10% of cases, decreases very markedly when ivabradine is added, this also being the case for headaches.

1. A composition comprising a combination of ivabradine, 3-{3-[{[(7S)-3,4-dimethoxybicyclo[4.2.0]octa-1,3,5-trien-7-yl]methyl}(methyl)amino]propyl}-7,8-dimethoxy-1,3,4,5-tetrahydro-2H-3-benzazepin-2-one, or addition salts thereof with a pharmaceutically acceptable acid and a calcium inhibitor selected from the dihydropyridine class.

2. The composition of claim 1, wherein the calcium inhibitor is amlodipine or addition salts thereof with a pharmaceutically acceptable acid.

3. The composition of claim 1, wherein the calcium inhibitor is amlodipine besylate.

4. The composition of claim 1, which comprises ivabradine hydrochloride and amlodipine besylate.

5. A pharmaceutical composition comprising as active ingredient a composition of claim 1, alone or in combination with one or more pharmaceutically acceptable excipients.

6. The pharmaceutical composition of claim 5, comprising as active ingredient a composition comprising a combination of ivabradine hydrochloride and amlodipine besylate alone or in combination with one or more pharmaceutically acceptable excipients.

7. A method for treating a living animal body, including a human, afflicted with angina, comprising the step of administering to the living animal body, including a human, an amount of the composition of claim 1 which is effective for treatment of angina.
